(19) United States
(12) Patent Application Publication
(10) Pub. No.: US 2013/0079667 A1
(43) Pub. Date: Mar. 28, 2013

(54) FLOW SENSOR WITH MEMS SENSING DEVICE AND METHOD FOR USING SAME

(71) Applicants: Ertugrul Berkcan, Clifton Park, NY (US); Stanton Earl Weaver, Broadalbin, NY (US); Shankar Chandrasekaran, Bangalore (IN); Nannan Chen, Clifton Park, NY (US)

(72) Inventors: Ertugrul Berkcan, Clifton Park, NY (US); Stanton Earl Weaver, Broadalbin, NY (US); Shankar Chandrasekaran, Bangalore (IN); Nannan Chen, Clifton Park, NY (US)

(73) Assignee: GENERAL ELECTRIC COMPANY, Schenectady, NY (US)

(21) Appl. No.: 13/247,107

(22) Filed: Sep. 28, 2011

(51) Int. Cl. A61M 16/00; A61B 7/00; G06F 19/00; B23P 11/00; G01F 1/00; G01F 1/34

(52) U.S. Cl. 600/586; 702/45; 702/47; 29/428; 128/204.23

(57) ABSTRACT

A flow sensor assembly, snore detection assembly, and methods for fabricating the same. The flow sensor assembly includes a flow conduit for fluid flow, a flow disrupter for imparting a disturbance to the fluid flow, a first sensor responsive to the disturbance of the fluid flow and configured to generate signals responsive to the disturbance of the fluid flow, and a processor for determining a flow rate for the fluid flow through the flow conduit based on a first algorithm determining an amplitude of the fluid flow in a first flow regime and a second algorithm determining a frequency of the fluid flow in a second flow regime.

### FIELD

The invention relates to a flow sensor using a microelectromechanical sensing (MEMS) device, and more particularly, to a MEMS-based flow sensor for use in a ventilation apparatus, such as a continuous positive airway pressure (CPAP) machine or a variable positive airway pressure (VPAP) machine.

### BACKGROUND

Ventilation and respiration machines have been used for many years in hospitals, assisted living quarters, and other locations. Respiratory ailments and issues continue to abound, rendering such machines a continuing necessity.

Further, a large percentage of the population suffers from some form of respiratory issue during sleep, such as, for example, sleep apnea. For example, it is estimated that between four and nine percent of middle-aged men and between two and four percent of middle-aged women suffer from some form of sleep apnea. Many such sufferers utilize ventilation and/or respiratory machines to assist in their nighttime sleeping. Two types of such machines are a continuous positive airway pressure (CPAP) machine and a variable positive airway pressure (VPAP) machine.

It is important to be able to accurately determine the flow rate of ventilation and/or respiratory machines. Due to the complex nature of breathing and the change in direction and speed of air flow during breathing, it is very difficult to determine flow rates along a spectrum of flow regimes from a very low flow rate to a very high flow rate.

With some of these concerns in mind, an improved ventilation system and methodology would be welcome in the art.

### SUMMARY

An embodiment of the invention provides a flow sensor assembly. The flow sensor assembly includes a flow conduit configured to allow fluid flow, a flow disrupter configured to impart a disturbance to the fluid flow, a first sensor disposed within the flow conduit at a first position, the first sensor being responsive to the disturbance of the fluid flow and being configured to generate signals responsive to the disturbance of the fluid flow, and a processor operably connected to the first sensor, wherein the processor is configured to determine a flow rate for the fluid flow through the flow conduit based on a first algorithm determining an amplitude of the fluid flow in a first flow regime and a second algorithm determining a frequency of the fluid flow in a second flow regime.

An aspect of the flow sensor assembly embodiment provides a flow conduit configured to allow fluid flow, a flow disrupter configured to impart a disturbance to the fluid flow, wherein the flow disrupter comprises a first part separated from a second part by a flow separator, first and second sensors respectively disposed within the flow conduit at first and second positions which are symmetrically located relative to the flow disrupter, the sensors being responsive to the disturbance of the fluid flow and being configured to generate signals responsive to the disturbance of the fluid flow, and a processor operably connected to the sensors, wherein the processor is configured to determine a flow rate and a direction for the fluid flow through the flow conduit based on a first algorithm determining an amplitude of the fluid flow in a first flow regime and a second algorithm determining a frequency of the fluid flow in a second flow regime.

An embodiment of the invention provides a method for fabricating a ventilation assembly. The method includes providing a flow conduit configured to allow fluid flow, locating a flow disrupter within the flow conduit, the flow disrupter being configured to impart a disturbance to the fluid flow, disposing a first sensor within the flow conduit at a first position, the first sensor being responsive to the disturbance of the fluid flow and being configured to generate signals responsive to the disturbance of the fluid flow, and operably connecting a processor to the first sensor, wherein the processor is configured to determine a flow rate for the fluid flow through the flow conduit based on a first algorithm determining an amplitude of the fluid flow in a first flow regime and a second algorithm determining a frequency of the fluid flow in a second flow regime.

An embodiment of the invention provides a method for fabricating a snore detector. The method includes providing a flow conduit configured to allow fluid flow, locating a flow disrupter within the flow conduit, the flow disrupter being configured to impart a disturbance to the fluid flow, disposing a first sensor within the flow conduit at a first position and a second sensor within the flow conduit at a second position, the first and second sensors being responsive to snoring and the disturbance of the fluid flow and being configured to generate signals characteristic of snoring and the disturbance of the fluid flow, placing a fan in fluid communication with the flow conduit, wherein the fan is configured to be activated only upon the detected presence of snoring, placing a flexible tube in fluid communication with the fan, placing a mask in fluid communication with the flexible tube, wherein the mask is configured to be worn by a person, and operably connecting a processor to the first and second sensors, wherein the processor is configured to determine characteristics indicative of snoring

An embodiment of the invention provides a snore detecting assembly, which includes a flow conduit configured to allow fluid flow, a flow disrupter configured to impart a disturbance to the fluid flow, a first sensor disposed within the flow conduit at a first position and a second sensor disposed within the flow conduit at a second position, the first and second sensors being responsive to sound and to the disturbance of the fluid flow and being configured to generate signals characteristic of the sound and the disturbance of the fluid flow, and a processor operably connected to the first and second sensors, wherein the processor is configured to distinguish between signals characteristic of the disturbance to the fluid flow and signals characteristic of sound.

These and other features, aspects and advantages of the present invention may be further understood and/or illustrated when the following detailed description is considered along with the attached drawings.

### DESCRIPTION OF THE DRAWINGS

FIG. 1 is a schematic view of a flow sensor system in accordance with an embodiment of the invention.

FIG. 2 is a schematic view of a flow sensor system in accordance with an embodiment of the invention.

FIG. 3 is a perspective view of a printed circuit board anchored in a flow conduit in accordance with an embodiment of the invention.

FIG. 4 is a schematic view of a flow sensor system in accordance with an embodiment of the invention.

FIG. 5 is a schematic view of a flow sensor system in accordance with an embodiment of the invention.

FIG. 6 is a perspective view illustrating a printed circuit board and flow disrupter in accordance with an embodiment of the invention.

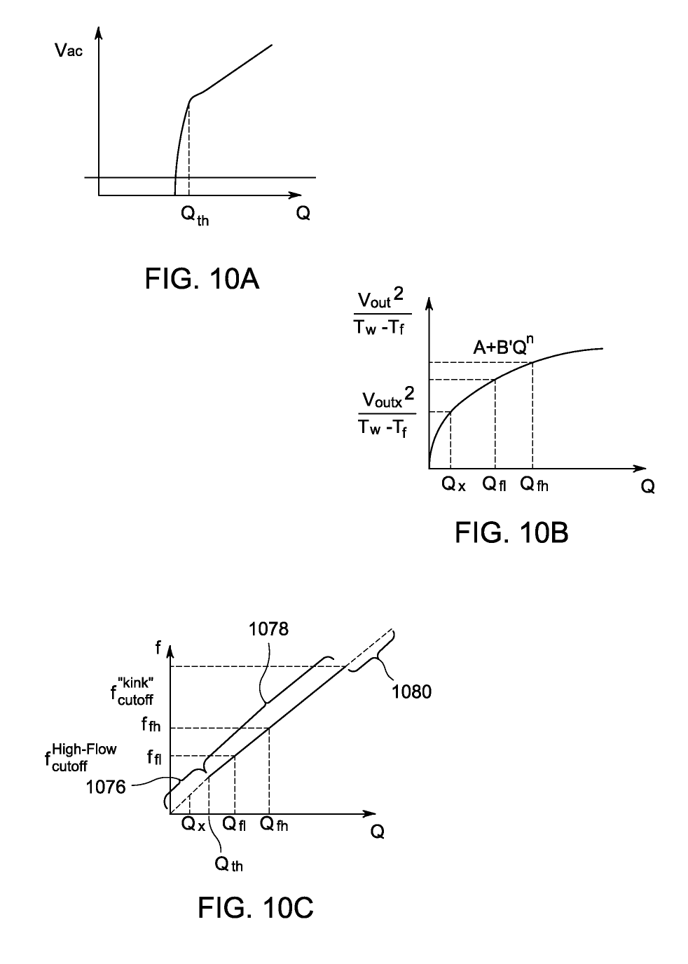

FIG. 7 is a perspective view illustrating an end of a flow conduit in accordance with an embodiment of the invention.

FIG. 8 is a schematic view of a ventilation apparatus in accordance with an embodiment of the invention.

FIG. 9 illustrates an electrical arrangement of a flow sensor system in accordance with an embodiment of the invention.

FIGS. 10A-10C are graphs charting three flow regimes in accordance with an embodiment of the invention.

FIGS. 11-17 are flow charts illustrating algorithms in accordance with embodiments of the invention.

### DETAILED DESCRIPTION

The present specification provides certain definitions and methods to better define the embodiments and aspects of the invention and to guide those of ordinary skill in the art in the practice of its fabrication. Provision, or lack of the provision, of a definition for a particular term or phrase is not meant to imply any particular importance, or lack thereof; rather, and unless otherwise noted, terms are to be understood according to conventional usage by those of ordinary skill in the relevant art.

Unless defined otherwise, technical and scientific terms used herein have the same meaning as is commonly understood by one of skill in the art to which this invention belongs. The terms “first”, “second”, and the like, as used herein do not denote any order, quantity, or importance, but rather are used to distinguish one element from another. Also, the terms “a” and “an” do not denote a limitation of quantity, but rather denote the presence of at least one of the referenced item, and the terms “front”, “back”, “bottom”, and/or “top”, unless otherwise noted, are merely used for convenience of description, and are not limited to any one position or spatial orientation. If ranges are disclosed, the endpoints of all ranges directed to the same component or property are inclusive and independently combinable (e.g., ranges of “up to about μwt. %, or, more specifically, about 5 wt. % to about 20 wt. %,” is inclusive of the endpoints and all intermediate values of the ranges of “about 5 wt. % to about 25 wt. %,” etc.).

The modifier “about” used in connection with a quantity is inclusive of the stated value and has the meaning dictated by the context (e.g., includes the degree of error associated with measurement of the particular quantity). Reference throughout the specification to “one embodiment”, “another embodiment”, “an embodiment”, and so forth, means that a particular element (e.g., feature, structure, and/or characteristic) described in connection with the embodiment is included in at least one embodiment described herein, and may or may not be present in other embodiments. In addition, it is to be understood that the described inventive features may be combined in any suitable manner in the various embodiments.

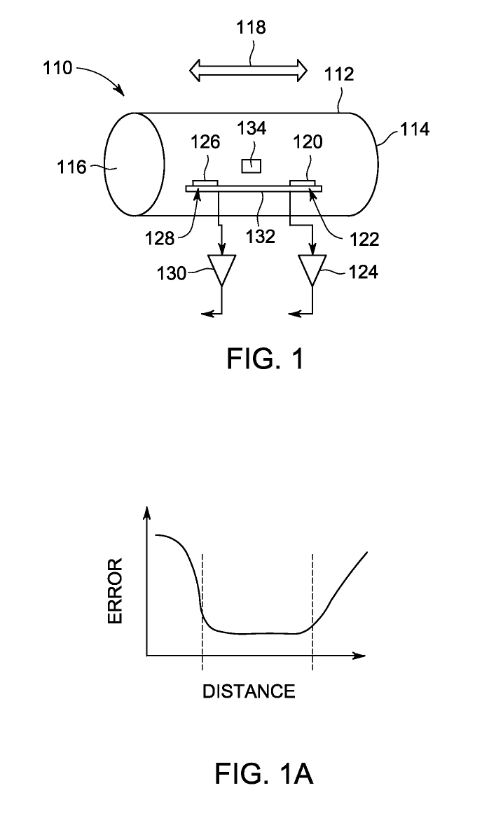

FIG. 1 illustrates schematically a flow sensor assembly 110 in accordance with an embodiment of the invention. The assembly 110 utilizes the principle that a disruption in a fluid flow creates certain characteristics, or vertices, that can be sensed and analyzed. For example, a fluid flow will have a certain direction, velocity, pressure, and temperature associated with it. By placing a disruption in the fluid stream, the velocity is altered, as are the pressure and temperature. These changes can be detected and analyzed to accurately determine the true fluid flow rate.

The assembly 110 includes a pair of sensing elements 120, 126. Each of the sensing elements 120, 126 is positioned within a conduit 112 that has an upstream opening 114 and a downstream opening 116. It should be understood that the terms “upstream” and “downstream” are relative terms that are related to the direction of flow 118. Thus, in some embodiments, if the direction of flow 118 extends from element 116 to element 114, then element 116 would be the upstream opening and element 114 would be the downstream element. For ease of description, the upstream side of the flow sensor assembly 110 will be the side closest to the opening 114 and the downstream side of the assembly will be the side closest to the opening 116.

A flow disrupter 134 is positioned equidistant between the sensing elements 120, 126. Further, the sensing elements 120, 126 are mounted on a printed circuit board (PCB) 132 at, respectively, first and second positions 122, 128. The purpose of the flow disrupter 134 is to form turbulence within the flow stream, such as, for example, waves or eddies. In so doing, the sensors 120, 126 can take measurements and send signals to, respectively signal conditioners 124, 130. The signal conditioners 124, 130 condition the signals by, for example, filtering or amplifying them, prior to sending the signals on to anti-aliasing filters and a processor (not shown) for analysis.

The locations of the first and second positions 122, 128, the shape of the flow disrupter 134, the positioning of the flow disrupter 134 relative to the sensors 120, 126 and within the conduit 112, and the size and positioning of the PCB 132 are all interrelated factors. For example, if the downstream sensor 126 is positioned too close to the flow disrupter 134, it will not pick up any of the turbulent vertices caused by the flow disrupter because it will be too far upstream to be able to detect the formation of such vertices. Conversely, if the downstream sensor 126 is positioned too far from the flow disrupter 134, it also will not pick up any of the turbulent vertices because they would have decayed to the point of being undetectable.

There are regions, located at a distance from the flow disrupter 134, at which the sensors 120, 126 are appropriately sited. These regions have a geometrical relationship wherein the error in the sensor reading is minimized. The relationship between error and the distance the sensor is from the flow disrupter is shown in the graph on FIG. 1. As shown, there is a region where the error of the sensor output is low and relatively unchanging. In one embodiment, the sensors 120, 126 are located equidistant from the flow disrupter 134. Although only one flow disrupter 134 is shown, in one embodiment two or more flow disrupters 134 may be utilized within a conduit 112.

The characteristics, or vertices, of flow that can be determined are flow speed, flow direction, the pressure of the flow, the temperature of the flow, the change in velocity of the flow, the change in pressure of the flow, and the heat transfer of the flow. Thus, the sensors 120, 126 can be any form of sensor capable of sensing any one or more of these vertices. For example, the sensing elements 120, 126 may be configured to determine pressure, temperature, change in pressure, change in temperature, or change in flow rate. In one embodiment, the sensors 120, 126 are pressure sensors. In another embodiment, the sensors 120, 126 are heaters. In yet another embodiment, the sensing elements 120, 126 are microelectromechanical devices.

The presence of two sensors 120, 126 is not necessary. A single sensor instead may be used. However, the presence of two sensors does provide certain benefits. For example, ascertaining the direction of a flow of fluid is impossible with a single sensor. Thus, for applications where determining the direction of flow is needed, two sensors would be required. Further, there is a certain amount of ambient noise in the turbulent flow of fluid. Signals from a single sensor cannot differentiate ambient noise from other noise caused by turbulence, and hence there may be more inherent error from a flow sensor apparatus having only one sensor. Signals from a pair of sensors, on the other hand, can parse out ambient noise from noise caused by the turbulence itself, thus decreasing the amount of error inherent in the analysis of the signals.

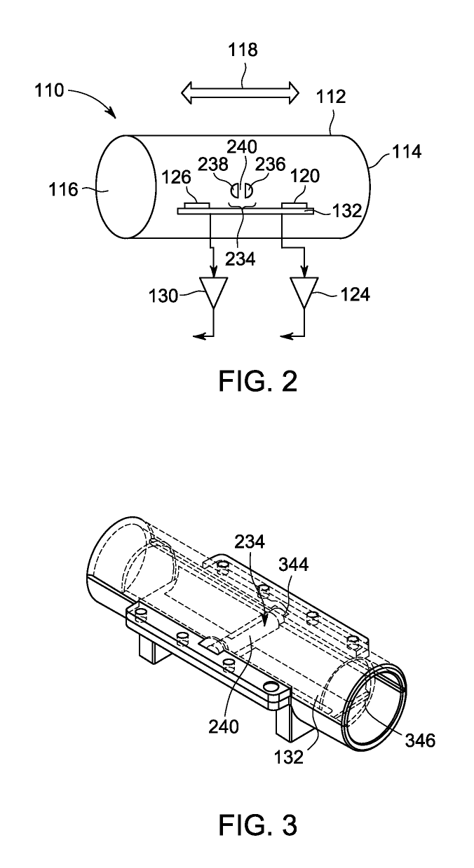

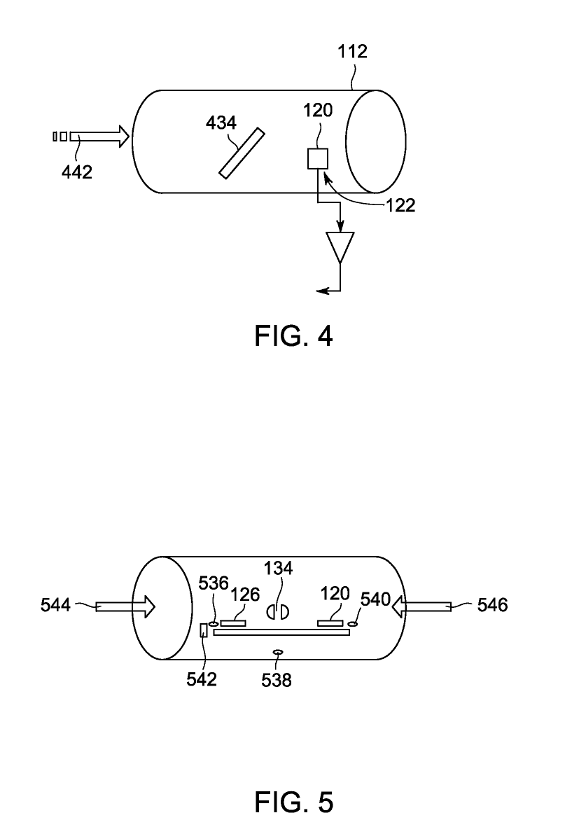

FIG. 2 illustrates the flow sensor assembly 110, but with a different flow disrupter 234. The flow disrupter 234 includes a first part 236 separated from a second part 238 by a flow separator 240. The first and second parts 236, 238 are blunt flow disrupters. Although shown as being separate elements, instead the first and second parts may be opposite sides of a single flow disrupter that has a flow separator portion eaten out of the middle portion (FIG. 3).

The flow disrupter 234 may be positioned orthogonal to the fluid flow direction through the conduit. For example, as shown in FIG. 3, the flow disrupter 234 may be anchored within ledges 344 on opposing sides of the conduit. Further, the PCB 132 may have arms 346 to allow it to be positioned properly within the conduit and anchored to sides of the conduit.

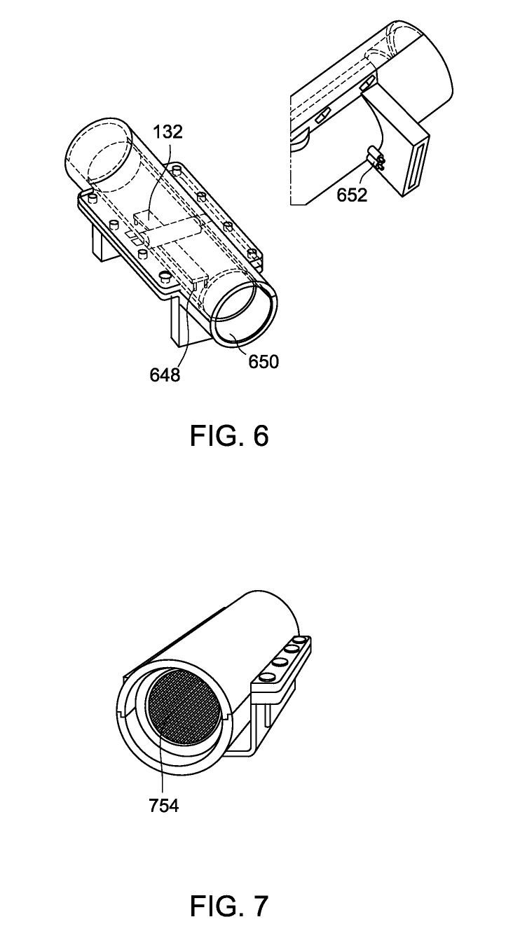

With specific reference to FIG. 4, there is shown a flow sensor assembly having a single sensor 120 and a planar flow disrupter 434. The fluid flow 442 hits the flow disrupter 434, which creates turbulent vertices in the fluid flow, which are in turn detected by the sensor 120 at position 122. The sensor 120 sends signals of the vertices through the signal conditioner and on to the processor (not shown). As indicated previously, such a system would have difficulty in rectifying signals of turbulent vertices from ambient noise within the flow stream. Further, such a system would likely be most useful in determining flow direction of the fluid flow 442.

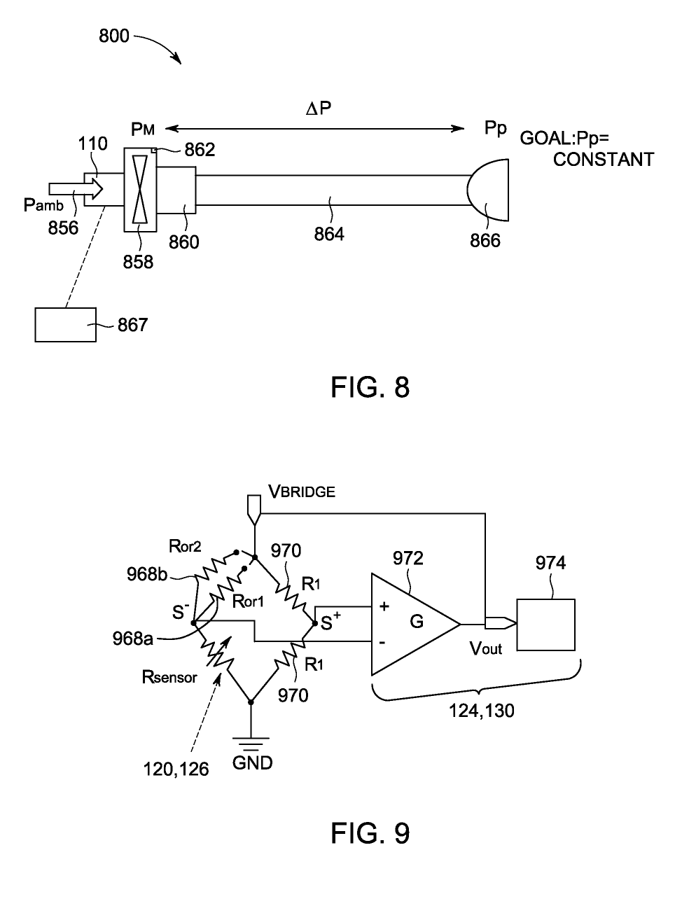

FIG. 5 illustrates additional embodiments of the invention. In one embodiment, two temperature sensors are provided. The temperature sensors can be any two of sensors 536, 538, and 540. The combination of two temperature sensors can determine the direction of flow as either being direction 544 or direction 546. If, for example, the direction of flow is direction 544, then the temperature sensor 536 will not pick up heat from the heater 126 but the temperature sensors 538, 540 will pick up heat from, respectively, the heater 126 and the heater 120. Thus, the discrepancy the amount of heat picked up by two of the temperature sensors 536, 538, 540 can determine the direction of flow.

Alternatively, a secondary flow disrupter 542 may be positioned near one of the sensors 120, 126. For one flow direction, the secondary flow disrupter will affect the DC values of one of the sensors, while in the opposite flow direction there will be no effect to the DC values of either of the sensors. For example, for a flow direction 544, the illustrated secondary flow disrupter 542 will affect the DC value of the sensor 126 but will not have an, or will have a negligible, effect on the sensor 120. For a flow direction 546, the illustrated secondary flow disrupter 542 will not affect the DC values of either sensor 120, 126.

In a third embodiment, direction of flow can be determined simply through the acknowledgement that the flow disrupter 134 will create, due to its presence, a higher flow downstream than is found upstream. Thus, the upstream sensor (126 for flow direction 544, 120 for flow direction 546) will record a lower flow rate than the downstream sensor.

While the PCB 132 may have arms as shown in FIG. 3, instead it may be anchored to a lower portion of the conduit through anchors 648. Signals from the PCB 132 and the sensors may be communicated from the conduit through electrical pins 652.

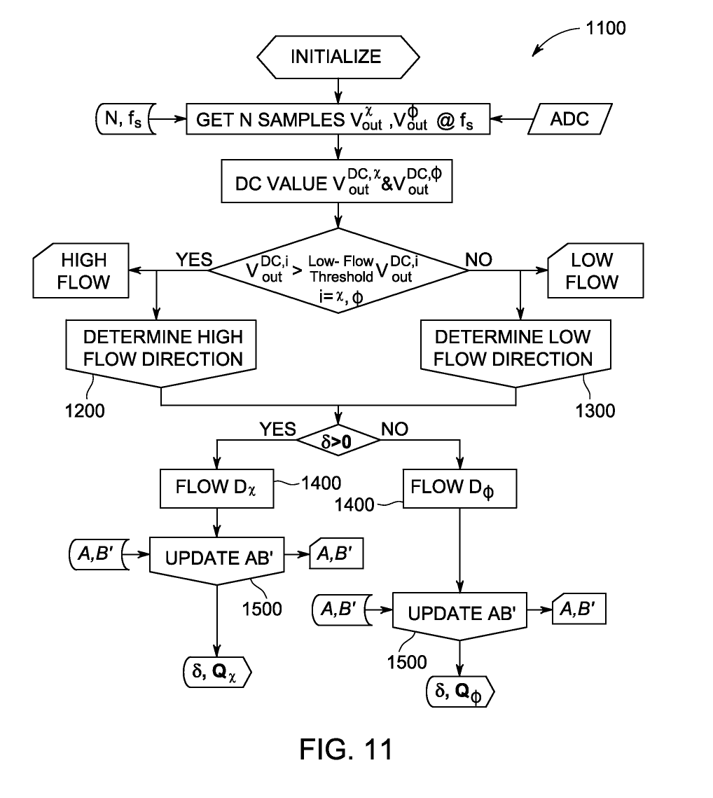

The conduit further may include a straightener section 650. The straightener section 650serves to condition the flow through the conduit. As illustrated in FIG. 7, the straightener section may include a screen 754 to assist in transitioning turbulent flow back into laminar flow.

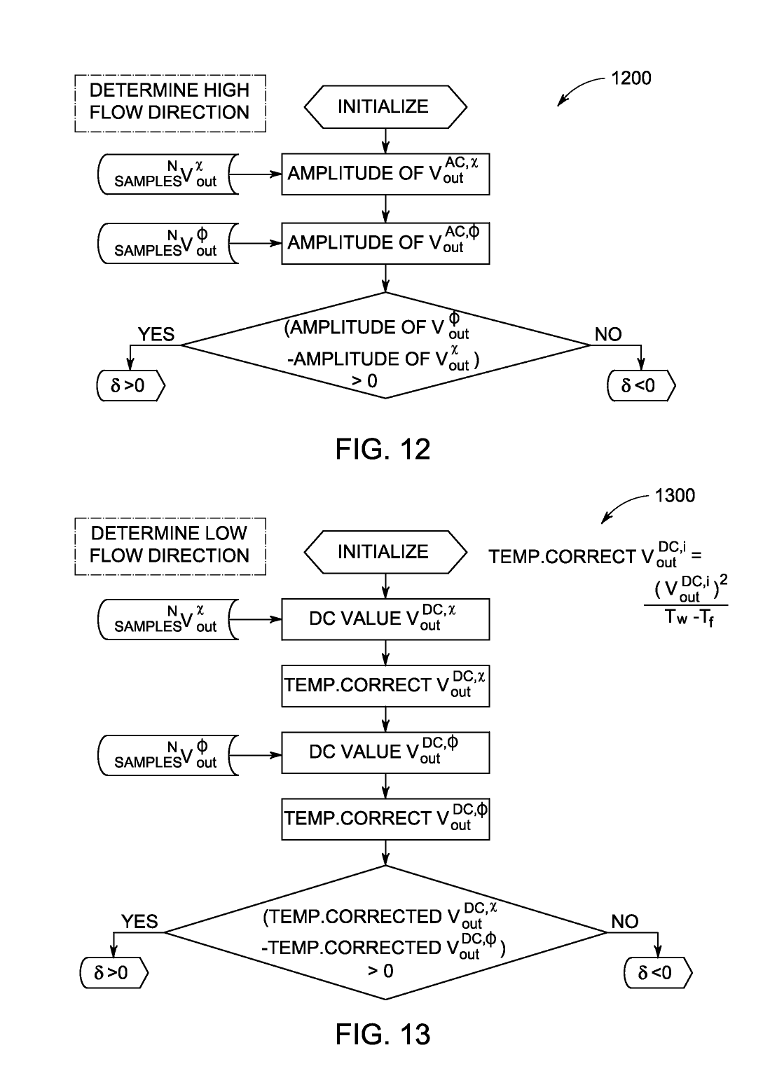

As illustrated schematically in FIG. 8, there is shown a ventilation assembly 800. The ventilation assembly may be, for example, a CPAP or a VPAP machine. The ventilation assembly 800 includes the flow sensor assembly 110, a fan 858, a tube 864, and a mask 866. Optionally, a humidifier 860 can be included upstream of the tube 864. In addition, a pressure sensor 862 may be located within the fan mechanism 858. While illustrated upstream of the fan 858, the flow sensor assembly 110 may instead be positioned further downstream, for example within the tube 864.

There is an ambient pressure P_{amb} in the fluid flow 856 entering the flow sensor assembly 110. The fan 858 is provided to create a higher pressure P_{M} that is used to facilitate the movement of a fluid through the tube 864 to the mask 866. There will be a pressure drop along the tube 864 between the higher pressure P_{M} at the fan 858 and the lower pressure P_{P} at the patient. A goal of the ventilation assembly 800 is to maintain a constant P_{P}. A processor 867 is provided to assist in that goal.

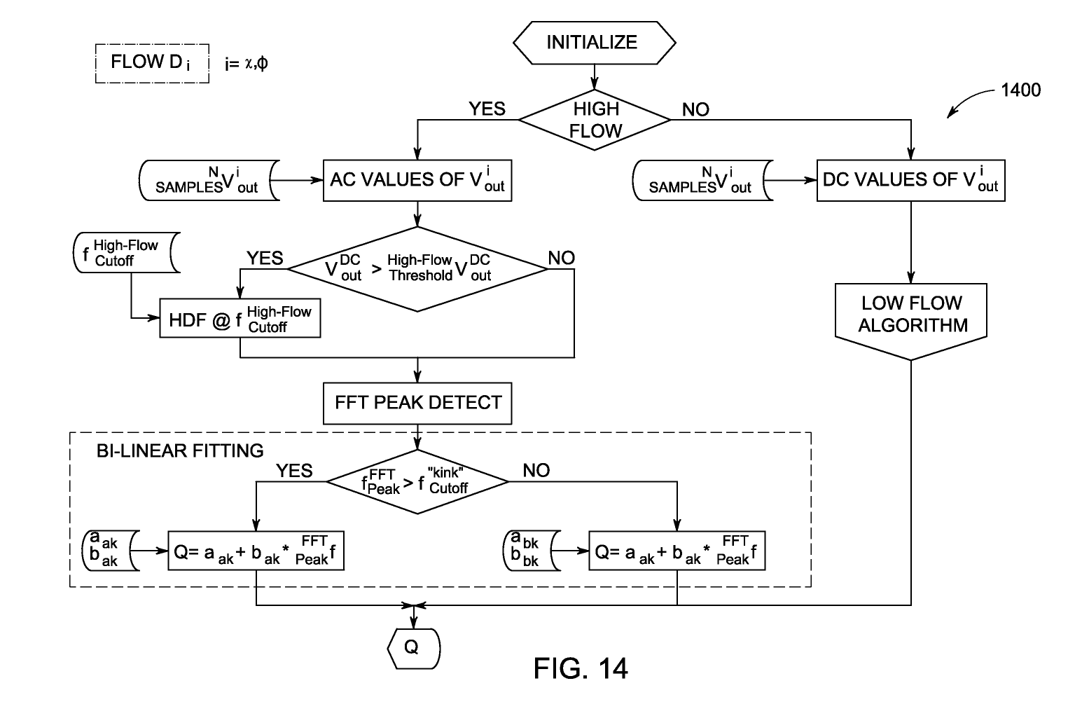

FIG. 9 illustrates the electrical circuitry of an exemplary flow sensor assembly 110. In this embodiment of the invention, the sensors 120, 126 are heaters, the electrical resistances of which are represented as the Rₛₑₙₛₒᵣ. The principle behind this electrical arrangement is to maintain the heaters 120, 126 at a particular temperature. This is accomplished through the use of two alternating overheat resistors Rₒᵣ₁ 968a and Rₒᵣ₂ 968b. The value of each of the overheat resistors Rₒᵣ₁ 968a and Rₒᵣ₂ 968b is intended to be greater than the ambient resistance of the Rₛₑₙₛₒᵣ. By switching between the overheat resistors Rₒᵣ₁ 968a and Rₒᵣ₂ 968b, the assembly can be run at different temperatures. At higher flow rates, for example, it is possible to obtain acceptable signal data from lower temperatures. Further, by running at different temperatures, it is possible to look at time constant and flow differential characteristics. The signals are moderated by identical resistors R₁ 970. Then, the signals are passed through the signal conditioners 124, 130, which are formed of a servo amplifier 972 and a signal conditioner 974, and forwarded on to the processor (not shown).

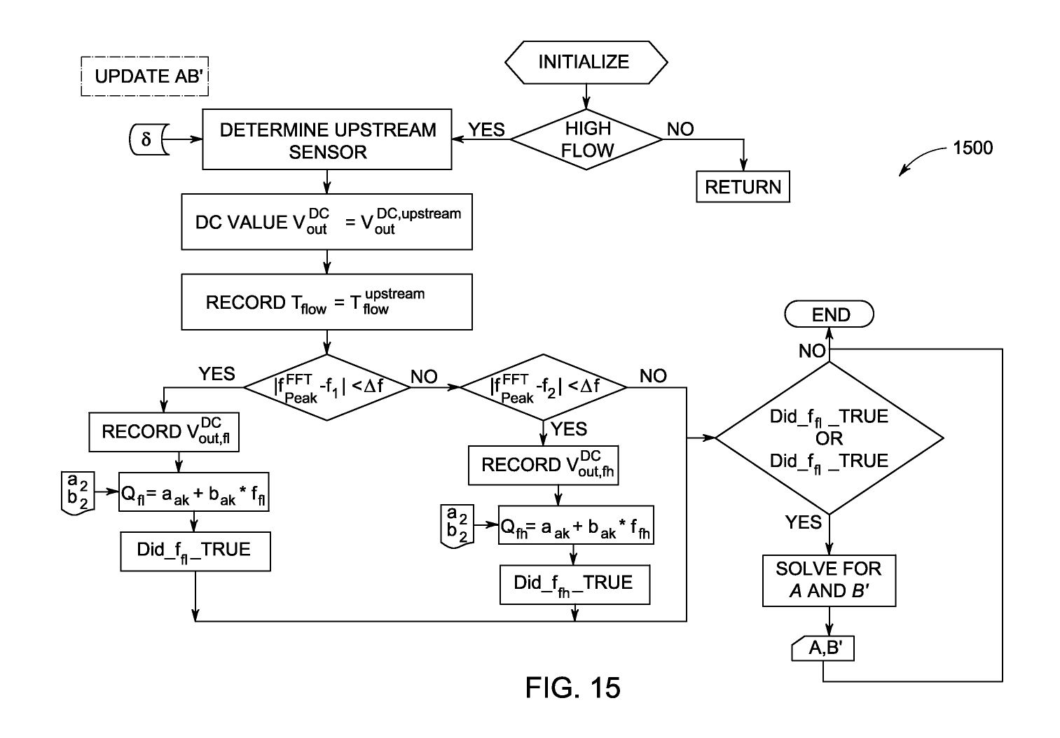

As noted before, in ventilation apparatuses the flow rate is constantly changing. For such apparatuses used to treat sleep apnea, for example, the rate of air will change from a high rate (during normal inhalation/exhalation) to a zero flow rate (during periods of time when the patient has stopped breathing). It has been determined that there are essentially three flow rate regimes that can be analyzed. As illustrated in FIG. 10C, a very low flow rate regime 1076 extends from a flow rate of zero to a threshold flow rate Qₜₕ. The threshold flow rate Qₜₕ is a flow rate at which vertices begin forming. In other words, it is the flow rate at which turbulence, and its vertices, can be detected by sensors. Above that flow rate there is a mid-flow regime 1078, followed by a high flow rate regime 1080. FIG. 10B illustrates the underlying characteristics of the algorithms used in embodiments of the invention. Specifically, FIG. 10B schematically illustrates the behavior of the flow amplitudes in the conduit at the very low flow rate regime 1076 and at the lower end of the mid-flow regime 1078.

FIG. 10A graphs the alternating current voltage V_{ac} of the sensors 120, 126 against flow rate Q. At very low flow rates, i.e., below Qₜₕ, the alternating current voltage V_{ac} rapidly increases over a small increase in flow rate. Once the mid-flow regime has been reached, i.e., above Qₜₕ, the alternating current voltage V_{ac} increases at a more linear relationship with an increase in the flow rate Q.

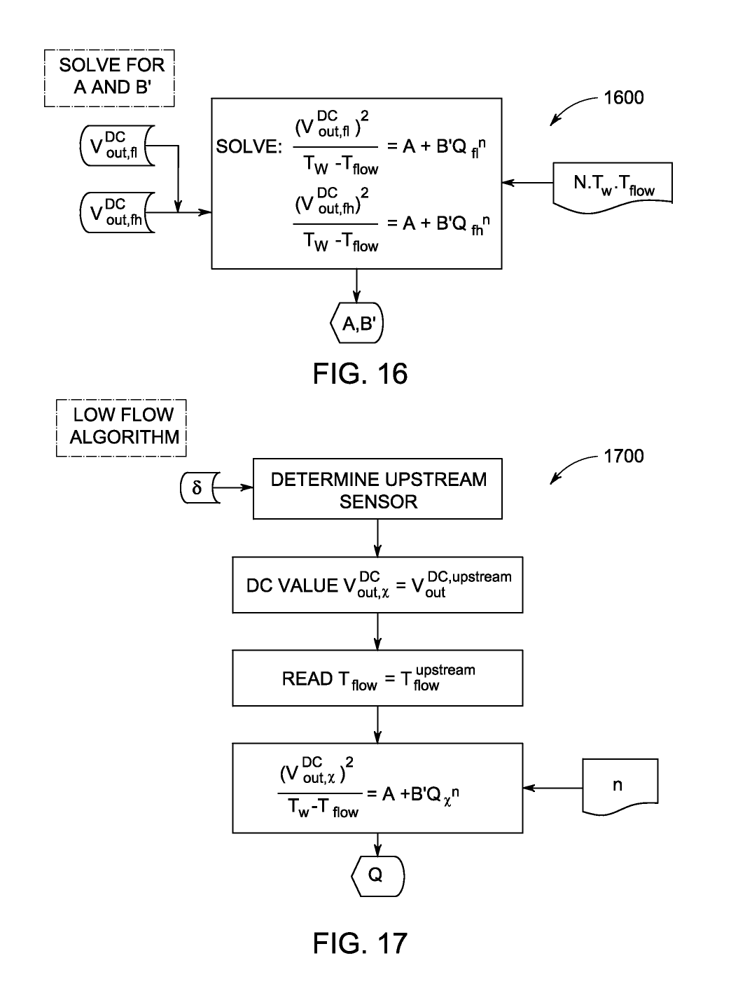

Next, with reference to FIGS. 11-17 will be described algorithms for accurate determination of fluid flow within a fluid flow assembly, such as the ventilation assembly 800.

FIG. 11 illustrates a decision tree 1100 for determining various flow variables for a flow sensor assembly, such as assembly 110. After initializing, a number N of samples are obtained. Specifically, samples of voltages V^{χ}ₒᵤₜ and V^{φ}ₒᵤₜ at a frequency fₛ are obtained. The voltage V^{χ}ₒᵤₜ denotes the output voltage read for one of the sensors 126, 120, while the voltage V^{φ}ₒᵤₜ denotes the output voltage read for the other of the sensors 126, 120. Then, the direct current values of voltages V^{DC,χ} and V^{DC,φ}ₒᵤₜ are obtained. Then, a determination is made whether the value of V^{DC,i}ₒᵤₜ is greater than the low-flow threshold V^{DC,i}ₒᵤₜ. If it is, then the flow is deemed to be high flow and the signals with a relationship with that high flow are sent to the high flow direction determination algorithm 1200. If, conversely, the value of V^{DC,i}ₒᵤₜ is not greater than the low-flow threshold V^{DC,i}ₒᵤₜ, then the flow is deemed to be low flow and the signals with a relationship with that low flow are sent to the low flow direction determination algorithm 1300.

Once the direction of the flow has been determined, either through the algorithm 1200 or the algorithm 1300, then a determination is made as to whether the direction of flow δ is greater than zero. If the direction of flow δ is greater than zero, then the flow of D_{χ} is determined by the flow Dᵢ algorithm 1400. If the direction of flow δ is not greater than zero, then the flow of D_{Φ} is determined by the flow Dᵢ algorithm 1400. Once the flow of D₁₀₂ is determined, then the AB′ for the flow of D_{χ} is updated by algorithm 1500 and δ and the flow rate for the flow of D_{χ} (Q_{χ}) are determined. Once the flow of D_{φ} is determined, then the AB′ for the flow of D_{φ}, which is determined by algorithm 1600 of FIG. 16, is updated by algorithm 1500 and δ and the flow rate for the flow of D_{φ} (Q_{φ}) are determined.

Algorithm 1200 determines the direction of a high flow regime of flow. Upon initialization, an amplitude of the voltage V^{AC,χ}ₒᵤₜ of the signal, determined from N number of samples of V^{χ}ₒᵤₜ taken by the sensors 120, 126, is obtained. Also, an amplitude of the voltage V^{AC,φ}ₒᵤₜ of the signal, determined from N number of samples of V^{φ}ₒᵤₜ taken by the sensors 120, 126, is obtained. Then, a determination is made as to whether the amplitude of the voltage V^{φ}ₒᵤₜ minus the amplitude of V^{χ}ₒᵤₜ is greater or less than zero. If greater than zero, then the flow of D_{χ} is determined by the flow Dᵢ algorithm 1400. If not greater than zero, then the flow of D_{φ} is determined by the flow Dᵢ algorithm 1400.

Algorithm 1300 determines the direction of a low flow regime of flow. Upon initialization, a direct current value of the voltage V^{DC,χ}ₒᵤₜ of the signal, determined from N number of samples of V^{χ}ₒᵤₜ taken by the sensors 120, 126, is obtained. Also, an a temperature corrected voltage V^{DC,χ}ₒᵤₜ is determined. Then, a direct current value of the voltage V^{DC,φ}ₒᵤₜ of the signal, determined from N number of samples of V^{φ}ₒᵤₜ taken by the sensors 120, 126, is obtained. A temperature corrected voltage V^{DC,φ}ₒᵤₜ is also determined. Then, a determination is made as to whether the temperature corrected voltage V^{DC,χ}ₒᵤₜ minus the temperature corrected voltage V^{DC,φ}ₒᵤₜ is greater or less than zero. If greater than zero, then the flow of D_{χ} is determined by the flow Dᵢ algorithm 1400. If not greater than zero, then the flow of D_{φ} is determined by the flow Dᵢ algorithm 1400.

In algorithm 1400, after initialization a determination is made as to whether the signals represent high flow, for example, the very high flow regime 1080 (FIG. 10C). If they do not represent high flow, then N number of samples of the voltage Vⁱₒᵤₜ are taken to determine the direct current values of the voltage Vⁱₒᵤₜ. Those values are then input into the low flow direction algorithm 1300. If instead they do represent high flow, then N number of samples of the voltage Vⁱₒᵤₜ are taken to determine the alternating current values of the voltage Vⁱₒᵤₜ. Then, a determination is made as to whether the voltage V^{DC}ₒᵤₜ is greater than the high-flow threshold voltage V^{DC}ₒᵤₜ. If it is not, then a fast Fourier transform peak detection is performed. If it is, then a high pass filter at a frequency f^{high-flow cutoff} is performed to weed out lower frequency interfering peaks, and then a fast Fourier transform peak detection is performed to find the peak for the high flow rate.

The fast Fourier transform peak detection is performed through bi-linear fitting. In FIG. 10C, for example, a linear slope is provided to schematically represent the flow regimes 1076, 1078, and 1080. In actuality, there may be some subtle kinks in the flow data such that a pair of sloped lines starting from the origin and steadily departing from one another may be a more appropriate graphing technique for the flow data. In bi-linear fitting, a determination is made as to whether a frequency f^{FFT}ₚₑₐₖ is greater than a frequency f^{“kink”}_{Cutoff}.

In update AB′ algorithm 1500, a high flow is determined. The update AB′ algorithm 1600 utilizes voltages for low flow V^{DC}_{out,fl} and voltages for high flow V^{DC}_{out,fh} to solve the following equations:

$$\begin{matrix}\frac{{( {V}_{out , fl}^{DC} )}^{2}}{{T}_{w} - {T}_{flow}} = A + {B}^{′} {Q}_{fl}^{n} & Equation 1 \\ \frac{{( {V}_{out , fh}^{DC} )}^{2}}{{T}_{w} - {T}_{flow}} = A + {B}^{′} {Q}_{fh}^{n} & Equation 2\end{matrix}$$

In the two above equations, the left-hand sides of the equations contain variables that are either measured or otherwise known through calibration techniques. Further, the low flow Q of Equation 1 and the high flow Q of Equation 2 are also known. Thus, there are two equations with two unknowns, namely A and B′, allowing for the solving of both unknowns in near real-time. Knowing A and B′ in near real-time allows for those values to be plugged into the algorithm 1700 to solve for Q.

In an alternative embodiment, the equations to be solved for in algorithm 1600 include a more explicit temperature correction. Specifically, the equations to be solved for in algorithm 1600 may be:

V_{out,fl}^{DC}+γT_{flow,fl}=A+B′Qⁿ_{fl} Equation 3:

V_{out,fh}^{DC}+γT_{flow,fh}=A+B′Q_{fh}ⁿ Equation 4:

Temperature corrected values assist in providing a more accurate assessment of flow rates.

In another embodiment, the equations to be solved in algorithm 1600 are altered to include a nth order polynomial. Specifically, the equations to be solved in algorithm 1600 may be:

$$\begin{matrix}\frac{{( {V}_{out , fl}^{DC} )}^{2}}{{T}_{w} - {T}_{flow}} = α + {β}_{1 fl} {Q}_{fl}^{n} + {β}_{2 fl} {Q}_{fl}^{2} + {β}_{3 fl} {Q}_{fl}^{3} + … & Equation 5 \\ \frac{{( {V}_{out , fh}^{DC} )}^{2}}{{T}_{w} - {T}_{flow}} = α + {β}_{1 fh} {Q}_{fh}^{n} + {β}_{2 fh} {Q}_{fh}^{2} + {β}_{3 fh} {Q}_{fh}^{3} + … & Equation 6\end{matrix}$$

Another embodiment of the invention includes a rapid response to changes in flow rates. By “rapid response” is meant a response that occurs within ten milliseconds of a change across an entire dynamic range in a flow rate. If the rapid response embodiment is incorporate within a CPAP machine, for example, the importance of such a response is fairly evident. Upon a patient entering a pattern where his breathing is disrupted, a rapid response, i.e., activation of a fan, would create a rapid change in the CPAP operation in response to the change in breathing pattern.

The rapid response to changes in the flow rate can be accomplished in several ways. For example, in one aspect, the frequency of the flow rate can be calculated, using a fast Fourier transform, to ascertain a rapid change in flow rates.

Alternatively, the amplitude of the signals from the sensors. By reviewing the output of the sensors, the amplitude of the signals can be ascertained. If a large amplitude change is seen, then a presumption can be made that the flow rate may be changing quickly. Any one of Equations 1-6 can be utilized to determine flow rates based on the sensors alone, and then subsequent flow rates as determined by the sensors can be reviewed. Once the determined flow rates from the sensors approach the flow rates calculated using fast Fourier transforms (FFT), FFTs can be used from that point on to continue tracking the changing flow rates.

Alternatively, two FFTs can be run in parallel. One FFT run would be the normal, long FFT. The other FFT would be a quick one using only the most recent values. For example, the long FFT may utilize 4,096 separate points of data in its calculations, while the quick FFT may only utilize 512 points. If the flow rate changes rapidly, the quick FFT will provide good resolution.

In another embodiment, zero crossing based frequency determination is used instead of fast Fourier transforms. In yet another embodiment, a special noise reduction and averaging algorithm is used in addition to the zero crossing to render the noise vulnerability of the zero crossing based algorithms.

In yet another embodiment, a phase locked loop approach is used instead of the fast Fourier transforms for the demodulation and the determination of the flow velocity. In yet another embodiment, a double phase locked loop is used instead of single phase locked loop.

In yet another embodiment, an adaptive notch filter-based or Kalman filter-based signal processing method is used for the demodulation of the sensor signal and the determination of the flow velocity.

In yet another embodiment, time-resolved and frequency-resolved demodulation and determination of the flow rate is obtained by the use of wavelet transforms and wavelet analysis.

An embodiment of the invention utilizes the flow sensor system as a snore detection system. Referring once again to FIG. 1, as flow enters the first opening 116, for example, the sensor 126 will not detect any vertices in the flow, as it is upstream of the flow disrupter 134. The sensor 120, however, will detect vertices caused by the flow disrupter 134. Thus, the output of second sine generator 130 will be different than the output of first sine generator 124. Specifically, the output of first sine generator 124 will include a sine wave like or periodic characteristic of the vertices caused by the flow disrupter 134.

If the flow sensor assembly 110 is being used in a CPAP or VPAP machine, the sensors 126, 120 can further detect the sound of snoring. If the person using the flow sensor assembly 110 begins to snore, both of the sensors 126, 120 will detect the sound and the output of both sine generators 130, 124 will include a sine wave. Thus, the presence of a sine wave in both sine generators 130, 124 is indicative of snoring.

To cancel out the sound, the output of sine generator 130 can be subtracted from the output of sine generator 124 to arrive at the sine wave for just the vertices in the flow. Alternatively, one can analyze the output spectrum of the sine generator 130 to find the characteristic peaks of snoring, which are found in certain frequency ranges. The characteristic frequency peaks for snoring have been studied. See, for example, Beck, R., et al., The acoustic properties of snores, Eur. Respir. J., 8, p. 2120-2128 (1995); Dalmasso, F., et al., Snoring: analysis, measurement, clinical implications and applications, Eur. Respir. J., 9, 146-159 (1996); Fiz, J. A., et al., Acoustic analysis of snoring sound in patients with simple snoring and obstructive sleep apnoea, Eur. Respir. J., 9, p. 2365-2370 (1996); Quinn, S. J., et al., The differentiation of snoring mechanisms using sound analysis, Clinical Otolaryngology & Allied Sciences, V. 21, I. 2, 119-123 (April 2007); Schäfera, J., et al., Digital signal analysis of snoring sounds in children, Int'l J. of Pediatric Otorhinolaryngology, V. 20, I. 3, 193-202 (December 1990); Saunders, N. C., et al., Is acoustic analysis of snoring an alternative to sleep nasendoscopy?, Clinical Otolaryngology & Allied Sciences, V. 29, I. 3, 242-246 (June 2004); and Agrawal, S., et al., Sound frequency analysis and the site of snoring in natural and induced sleep, Clinical Otolaryngology & Allied Sciences, V. 27, I. 3, 162-166 (June 2002).

Conversely, since the signals of flow can be separated out from the signals of snoring, the signals of snoring can be isolated and looked for. Specifically, by adding the outputs of the two sine generators 130, 124 and then subtracting out the absolute value of the difference of the outputs of the two sine generators 130, 124,

(130ₒᵤₜ+124ₒᵤₜ)−|130ₒᵤₜ−124ₒᵤₜ|

the result are the signals for sound, i.e., snoring.

Since the signals for snoring can be isolated out, a processor 867 (FIG. 8) for a CPAP or VPAP machine can provide refined functions. For example, the processor can provide increased pressure or can modulate the pressure in response to the signals for snoring. Further, the processor can, for example, start the fan, such as fan 858 (FIG. 8), in response to snoring. Alternatively, the processor can turn off the fan 858 in response to no snoring signals being detected.

While the invention has been described in detail in connection with only a limited number of embodiments, it should be readily understood that the invention is not limited to such disclosed embodiments. Rather, the invention can be modified to incorporate any number of variations, alterations, substitutions or equivalent arrangements not heretofore described, but which are commensurate with the spirit and scope of the invention. For example, while embodiments have been described in terms that may initially connote singularity, it should be appreciated that multiple components may be utilized. Additionally, while various embodiments of the invention have been described, it is to be understood that aspects of the invention may include only some of the described embodiments. Accordingly, the invention is not to be seen as limited by the foregoing description, but is only limited by the scope of the appended claims.

What is claimed as new and desired to be protected by Letters Patent of the United States is:

1. A flow sensor assembly, comprising:
a flow conduit configured to allow fluid flow;
a flow disrupter configured to impart a disturbance to the fluid flow;
a first sensor disposed within the flow conduit at a first position, said first sensor being responsive to the disturbance of the fluid flow and being configured to generate signals responsive to the disturbance of the fluid flow; and
a processor operably connected to said first sensor, wherein said processor is configured to determine a flow rate for the fluid flow through said flow conduit based on a first algorithm determining an amplitude of the fluid flow in a first flow regime and a second algorithm determining a frequency of the fluid flow in a second flow regime.

2. The flow sensor assembly of claim 1, wherein the flow disrupter comprises a blunt flow disrupter or a planar flow disrupter.

3. The flow sensor assembly of claim 2, wherein the blunt flow disrupter comprises a first part separated from a second part by a flow separator.

4. The flow sensor assembly of claim 1, wherein the first sensor is a microelectromechanical sensor.

5. The flow sensor assembly of claim 1, comprising a second sensor disposed within the flow conduit at a second position.

6. The flow sensor assembly of claim 5, wherein the second sensor is a microelectromechanical sensor.

7. The flow sensor assembly of claim 5, wherein the first and second positions are symmetrically located relative to the flow disrupter.

8. The flow sensor assembly of claim 5, wherein the processor is configured to determine a flow direction for the fluid flow through said flow conduit.

9. The flow sensor assembly of claim 5, comprising a second flow disrupter.

10. The flow sensor assembly of claim 1, comprising electrical pins extending from the processor through the flow conduit.

11. The flow sensor assembly of claim 1, wherein the processor is configured to compute a modified fast Fourier transform (FFT) function of the signals responsive to the disturbance of the fluid flow generated by said sensors and the differences between the signals responsive to the disturbance of the fluid flow.

12. The flow sensor assembly of claim 1, wherein the first flow regime has a flow rate less than the second flow regime.

13. The flow sensor assembly of claim 1 for use within a ventilation assembly.

14. The flow sensor assembly of claim 13, wherein the ventilation assembly comprises a continuous positive airway pressure (CPAP) machine or a variable positive airway pressure (VPAP) machine.

15. The flow sensor assembly of claim 14, wherein the ventilation assembly comprises:
a fan in fluid connection with the flow sensor assembly;
a flexible tube in fluid connection with the fan; and
a mask in fluid connection with the flexible tube.

16. The flow sensor assembly of claim 15, wherein the fan is configured to be activated only upon the detected presence of snoring.

17. The flow sensor assembly of claim 15, wherein the fan is activated, in response to a rapid change in the fluid flow, within ten milliseconds.

18. A flow sensor assembly, comprising:
a flow conduit configured to allow fluid flow;
a flow disrupter configured to impart a disturbance to the fluid flow, wherein the flow disrupter comprises a first part separated from a second part by a flow separator;
first and second sensors respectively disposed within the flow conduit at first and second positions which are symmetrically located relative to the flow disrupter, said sensors being responsive to the disturbance of the fluid flow and being configured to generate signals responsive to the disturbance of the fluid flow; and
a processor operably connected to said sensors, wherein said processor is configured to determine a flow rate and a direction for the fluid flow through said flow conduit based on a first algorithm determining an amplitude of the fluid flow in a first flow regime and a second algorithm determining a frequency of the fluid flow in a second flow regime.

19. The flow sensor assembly of claim 18, wherein the processor is configured to compute a modified fast Fourier transform (FFT) function of the signals responsive to the disturbance of the fluid flow generated by said sensors and the differences between the signals responsive to the disturbance of the fluid flow.

20. The flow sensor assembly of claim 18, wherein the first flow regime has a flow rate less than the second flow regime.

21. The flow sensor assembly of claim 18 for use within a ventilation assembly.

22. The flow sensor assembly of claim 21, wherein the ventilation assembly comprises a continuous positive airway pressure (CPAP) machine or a variable positive airway pressure (VPAP) machine.

23. The flow sensor assembly of claim 22, wherein the ventilation assembly comprises:
a fan in fluid connection with the flow sensor assembly;
a flexible tube in fluid connection with the fan; and
a mask in fluid connection with the flexible tube.

24. The flow sensor assembly of claim 23, wherein the fan is configured to be activated only upon the detected presence of snoring.

25. The flow sensor assembly of claim 23, wherein the fan is activated, in response to a rapid change in the fluid flow, within ten milliseconds.

26. A method for fabricating a ventilation assembly, comprising:
providing a flow conduit configured to allow fluid flow;
locating a flow disrupter within the flow conduit, the flow disrupter being configured to impart a disturbance to the fluid flow;
disposing a first sensor within the flow conduit at a first position, the first sensor being responsive to the disturbance of the fluid flow and being configured to generate signals responsive to the disturbance of the fluid flow; and
operably connecting a processor to the first sensor, wherein the processor is configured to determine a flow rate for the fluid flow through the flow conduit based on a first algorithm determining an amplitude of the fluid flow in a first flow regime and a second algorithm determining a frequency of the fluid flow in a second flow regime.

27. The method of claim 26, wherein said locating a flow disrupter within the flow conduit comprises locating a blunt flow disrupter having a first part separated from a second part by a flow separator or locating a planar flow disrupter.

28. The method of claim 26, comprising disposing a second sensor within the flow conduit at a second position, wherein one of the first and second positions is located upstream of the flow disrupter and the other of the first and second positions is located downstream of the flow disrupter.

29. The method of claim 28, comprising operably connecting the processor to the second sensor, the processor being configured to determine a direction of the fluid flow through the flow conduit.

30. The method of claim 26, comprising operably connecting the processor with a data storage unit for storing data obtained from the processor.

31. A method for fabricating a snore detector, comprising:
providing a flow conduit configured to allow fluid flow;
locating a flow disrupter within the flow conduit, the flow disrupter being configured to impart a disturbance to the fluid flow;
disposing a first sensor within the flow conduit at a first position and a second sensor within the flow conduit at a second position, the first and second sensors being responsive to snoring and the disturbance of the fluid flow and being configured to generate signals characteristic of snoring and the disturbance of the fluid flow;
placing a fan in fluid communication with the flow conduit, wherein the fan is configured to be activated only upon the detected presence of snoring;
placing a flexible tube in fluid communication with the fan;
placing a mask in fluid communication with the flexible tube, wherein the mask is configured to be worn by a person; and
operably connecting a processor to the first and second sensors, wherein the processor is configured to determine characteristics indicative of snoring.

32. The method of claim 31, comprising operably connecting the processor with a data storage unit for storing data obtained from the processor.

33. The method of claim 31, wherein the processor is configured to isolate the signals characteristic of snoring from the signals characteristic of the disturbance of the fluid flow.

34. A snore detecting assembly, comprising:
a flow conduit configured to allow fluid flow;
a flow disrupter configured to impart a disturbance to the fluid flow;
a first sensor disposed within the flow conduit at a first position and a second sensor disposed within the flow conduit at a second position, said first and second sensors being responsive to sound and to the disturbance of the fluid flow and being configured to generate signals characteristic of the sound and the disturbance of the fluid flow; and
a processor operably connected to said first and second sensors, wherein said processor is configured to distinguish between signals characteristic of the disturbance to the fluid flow and signals characteristic of sound.

35. The snore detecting assembly of claim 34, wherein the flow disrupter comprises a blunt flow disrupter or a planar flow disrupter.

36. The snore detecting assembly of claim 35, wherein the blunt flow disrupter comprises a first part separated from a second part by a flow separator.

37. The snore detecting assembly of claim 34, wherein the first and second sensors are a microelectromechanical sensors.

38. The snore detecting assembly of claim 34, wherein the processor is configured to isolate the signals characteristic of the sound from the signals characteristic of the disturbance of the fluid flow.

39. The snore detecting assembly of claim 38, comprising:
a fan in fluid connection with the flow conduit;
a flexible tube in fluid connection with the fan; and
a mask in fluid connection with the flexible tube.

40. The snore detecting assembly of claim 39, wherein the processor is configured to start the fan in response to the signals responsive to the sound.

41. The snore detecting assembly of claim 40, wherein the processor is configured to stop the fan in response to an absence of the signals responsive to the sound.
